(19) United States
(12) Patent Application Publication
(10) Pub. No.: US 2023/0107274 A1
(43) Pub. Date: Apr. 6, 2023

(54) AN ORAL PHARMACEUTICAL COMPOSITION COMPRISING AN EXTRACT OF COMBINED HERBS COMPRISING LONGANAE ARILLUS FOR THE TREATMENT OR ALLEVIATION OF INFLAMMATORY DISEASE AND THE USE THEREOF

(71) Applicants: Ok Nam PARK, Seoul (KR); MEDIHELPLINE CO., LTD, Seoul (KR)

(72) Inventors: Ok Nam PARK, Seoul (KR); Hyi In RYU, Seoul (KR)

(21) Appl. No.: 17/911,401

(22) PCT Filed: Mar. 25, 2021
(86) PCT No.: PCT/KR2021/003728

§ 371 Date: Sep. 13, 2022

(30) Foreign Application Priority Data
Apr. 3, 2020 (KR) 10-2020-0040949
Mar. 12, 2021 (KR) 10-2021-0032875

(51) Int. Cl. A61K 36/77; A61K 9/00; A61K 36/236; A61K 36/69; A61P 29/00; A61P 19/02; A61K 47/02; A61K 47/14; A61K 9/14; A61K 9/20; A61K 9/48; A23L 33/105; A23L 2/52

(52) U.S. Cl. CPC A61K 36/77; A61K 9/0053; A61K 36/236; A61K 36/69; A61P 29/00; A61P 19/02; A61K 9/0019; A61K 47/02; A61K 47/14; A61K 9/146; A61K 9/145; A61K 9/143; A61K 9/2059; A61K 9/2018; A61K 9/2013; A61K 9/4858; A61K 9/485; A61K 9/4866; A61K 9/0095; A23L 33/105; A23L 2/52; A23V 2002/00

(57) ABSTRACT

The present invention relates to an oral pharmaceutical composition comprising a combined herb extract of Longanae Arillus, Ligustici Tenuissimi Rhizoma and Polygalae radix, the present inventors demonstrated that the anti-inflammatory/anti-rheumatic effects of inventive combined composition is potent by accomplishing in vitro experiments such as the inhibitory test on the expression of cytokines involved in inflammation (RPLPO, TSLP, GM-CSF and IL-1beta) (Experimental Example 1); Cell viability test on HT-29 and THP-1 cell (in vitro Experimental Example 2); Anti-inflammatory activity in THP-1 cell (in vitro, Experimental Example 3); inhibitory effect on autophagy activity (in vitro, Experimental Example 4.) as well as in vivo experiments such as inhibitory effect on arthritis sing by arthritis-induced rat animal model (in vivo, Experimental Example 5), therefore, it is confirmed that inventive combined extract is very useful in the alleviation or treatment of inflammatory disease and arthritis disease as a form of oral pharmaceutical composition.

### TECHNICAL FIELD

The present invention is related to an oral pharmaceutical composition comprising an extract of combined herbs comprising Longanae arillus for the treatment or prevention of inflammatory disease and the use thereof.

### BACKGROUND ART

Generally, an inflammatory response is a normal response of human body associated with an edema, a pain etc in case that a tissue or a cell received any invasion causing some organic change in the tissue or cell. Recently, various kinds of cytokines have been found to be involved in the inflammatory disease.

Therefore, many studies have been performed to develop effective drugs to inhibit the production of various cytokines such as IL-4, IL-5, and IL-13, and immunoglobulin E etc, which are involved in the activation of inflammatory cells resulting in leucotriene biosynthesis, a system secreted by inflammatory cells such as neutrophil etc and caused by the occurrence of inflammation, allergic response or asthma etc.

While the inflammatory phase continues to develop with pro-inflammatory cytokines such as TNF-α, IL-1β, IL-6 etc and MMPs (matrix metalloproteinases, such as PDGF, VEGF, and IGF, it is known that the expression of growth factor reduces (Trengove N J, Bielefeldt-Ohmann H, Stacey M C (2001) Mitogenic activity and cytokine levels in non-healing and healing chronic leg ulcers. Wound Repair and Regeneration. 8: 13-25.; Armstrong D G, Jude E B (2002) The Role of Matrix Metalloproteinases in Wound Healing. Journal of the American Podiatric Medical Association. 92: 12-18.).

MMPs (matrix metalloproteinases) are controlled by TIMPs (tissue inhibitors of metalloproteinases) in wound area, which decompose extracellular substrates, enabling re-epithelialization (Martins V L, Caley M, O'Toole E A (2013) Matrix metalloproteinases and epidermal wound repair. Cell and Tissue Research. 351: 255-268).

In particular, there have been focused in the research on MMP-9 which has been known to have the most harmful effects on chronic wounds among MMPs (Jones J I, Nguyen T T, Peng Z, Chang M (2019) Targeting MMP-9 in Diabetic Foot Ulcers. Pharmaceuticals. 12: 79.; Reiss M J, Han Y P, Garcia E, Goldberg M, Yu H, Garner W L (2010) Matrix metalloproteinase-9 delays wound healing in a murine wound model. Surgery. 147: 295-302.).

Accordingly, there has been still needed to develop more effective drug and cosmetics in treating and alleviating inflammatory diseases from natural source with low side effects than conventionally used drugs till now.

Longanae Arillus, a seed coat of Dimocarpus longan, Euphoria longan or the same species belonged to Sapindaceae has been reported to contain a glucose, fructose, protein etc and to show cardio-protective effect, appetite stimulating effect etc (Chung B. S et al, Dohaehyangyakdaesajeon, youngrimsa, 2^{nd} Ed. p 197-198, 1998).

Ligustici Tenuissimi Rhizoma, a rhizoma or root of Ligusticum tenuissimum Kitagawa, Ligusticum sinense Oliv, Ligusticum jeholense Nakai et Kitagawa or the same species belonged to Umbelliferae has been reported to contain a cnidilide, 3-butyl phthalide etc and to show anti-bacterial effect etc (Chung B. S et al, Dohaehyangyakdaesajeon, youngrimsa, 2^{nd} Ed. P428-429, 1998).

Polygalae radix, a root of Polygala tenuifolia Willd., or the same species belonged to Polygalaceae has been reported to contain various sanponis and to show expectorant activity, anti-bacterial effect etc (Chung B. S et al, Dohaehyangyakdaesajeon, youngrimsa, 2^{nd} Ed. P798-799, 1998).

However, there has been not reported or disclosed on the preventing or alleviating activity of an orally applied extract of combined herbs of Longanae Arillus, Ligustici Tenuissimi Rhizoma and Polygalae radix showing potent treating effect on inflammatory diseases in any of above cited literatures, and the disclosures of which are incorporated herein by reference.

### DISCLOSURE OF INVENTION

### Technical Problem

To investigate the anti-inflammatory effect of a combined herb extract of Longanae Arillus, Ligustici Tenuissimi Rhizoma and Polygalae radix, the inventors of present invention have intensively carried out various experiments including in vitro experiments such as the inhibitory test on the expression of cytokines involved in inflammation (RPLPO, TSLP, GM-CSF and IL-1beta) (Experimental Example 1); Cell viability test on HT-29 and THP-1 cell (in vitro Experimental Example 2); Anti-inflammatory activity in THP-1 cell (in vitro, Experimental Example 3); inhibitory effect on autophagy activity (in vitro, Experimental Example 4.) as well as in vivo experiments such as inhibitory effect on arthritis using by arthritis-induced rat animal model (in vivo, Experimental Example 5). As a result of these investigations, the inventors finally completed the present invention by confirming that inventive combined herb extract strongly inhibited and alleviated inflammatory diseases.

### Solution to Problem

The technical solution to solve the problem of the background art is for the development of novel herb formulation for treating and preventing inflammation disease or arthritis diseases.

Accordingly, it is an object of the present invention to provide an oral pharmaceutical composition comprising a combined herb extract of Longanae Arillus, Ligustici Tenuissimi Rhizoma and Polygalae radix as an active ingredient to treat and alleviate inflammatory diseases.

The term “combined herb extract” defined herein comprises the combined herb extract, i.e., (a) combined herb extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50:0.1-50 weight part (w/w), more preferably, 0.1-10:0.1-10:0.1-10 weight part (w/w), more and more preferably, 1-5:1-5:1-5 weight part (w/w), most preferably, 1-3:1-3:1-3 weight part (w/w); or (b) the combination of each extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50:0.1-50 weight part (w/w), more preferably, 0.1-10:0.1-10:0.1-10 weight part (w/w), more and more preferably, 1-5:1-5:1-5 weight part (w/w), most preferably, 1-3:1-3:1-3 weight part (w/w) in the present invention.

The term “extract” disclosed herein comprises the extract which can be extracted with at least one solvent selected from water, C₁-C₄ lower alkyl alcohol such as methanol, ethanol, propanol, butanol, etc, acetone, ethyl acetate, chloroform, hexane, butyleneglycol, propyleneglycol or glycerin, preferably, water, methanol, ethanol, more preferably, water or 10-90% (v/v) ethanol in water, most preferably, water or 20-80% (v/v) ethanol in water.

The term “inflammatory diseases” disclosed herein comprises the disease selected from group of pruritus caused by dermatitis, atopic dermatitis, conjunctivitis, periodontitis, rhinitis, middle ear infection, sore throat, tonsillitis, pneumonia, gastric ulcer, gastritis, Crohn's disease, colitis, hemorrhoids, gout, rheumatoid fever, lupus, fibromyalgia, tendinitis, tenosynovitis Peritendinitis, myositis hepatitis, cystitis, nephritis, Sjogren's syndrome, chronic inflammation and acute inflammation.

The term, “anti-Inflammation” disclosed herein, not limited thereto, means all the mechanism to inhibit various inflammation.

The term, “anti-rheumatic” disclosed herein, not limited thereto, means all the mechanism to inhibit various rheumatism.

Inflammation is part of the complex biological response of body tissues to harmful stimuli, such as pathogens, damaged cells, or irritants, and the nonspecific immune response such as heat, pain, redness, swelling, etc is called as “inflammatory response”

Inflammation can be classified as (a) acute inflammation, the initial response of the body to harmful stimuli, is achieved by the increased movement of plasma and leukocytes (especially granulocytes) from the blood into the injured tissues and then a series of biochemical events propagates and matures the inflammatory response, involving the local vascular system, the immune system, and various cells within the injured tissue and (b) Prolonged inflammation, known as chronic inflammation, leading to a progressive shift in the type of cells present at the site of inflammation, such as mononuclear cells, and being characterized by simultaneous destruction and healing of the tissue from the inflammatory process.

Generally, the macrophage in damaged cell excrete various cytokines, which activates T lymphocyte and mast cell, a lymphocyte, releases various histamines, which initiate internal barrier response, resulting in inducing inflammation of the inflected cells. Accordingly, the expressed level of cell cytokines may be used as an indicator of the activation of inflammatory response (the other aspects, anti-inflammatory activity). The “anti-inflammatory activity” disclosed herein denotes the inhibitory activity against various skin inflammation.

Cytokines means all the immunological substances including chemokines, interferons, interleukins, lymphokines, and tumour necrosis factors produced by a broad range of cells, including immune cells like macrophages, B lymphocytes, T lymphocytes and mast cells, as well as endothelial cells, fibroblasts, and various stromal cells, which are released through immunological progress caused by the infiltration of various pathogen such as virus etc.

Generally, cytokines are released at initial stage of infection, however, released constantly where the immune system becomes extraordinarily activated. When the high-level of cytokines are released for a long time such as more than week, we called as “a cytokine storm”, which is a physiological reaction in which the innate immune system causes an uncontrolled and excessive release of pro-inflammatory signaling molecules called cytokines and it exacerbates the inflammation resulting from the extremely abundant homing of immune cells to the inflected area, causes to blood extravasation through the loosening of blood vessel and severely to death. The term “the inhibitory activity of cytokine expression” disclosed herein can be interpreted as a prevention, treatment or improvement of cytokine storm.

The term “cytokine” disclosed herein, not intended to limit thereto, comprises various cytokine involved in dermatitis, such as atopic dermatitis, specifically, the cytokine selected from group of TLSP (thymic stromal lymphopoietin), colony stimulating factor (CSF) such as GM-CSF (granulocyte-macrophage colony stimulating factor), M-CSF (macrophage colony stimulating factor), G-CSF (granulocyte colony stimulating factor) and the like, interleukins such as interleukin-1 (IL-1), IL-4, IL-10, IL-12, IL-13, IL-31, IL-33 and the like, tumor necrosis factor alpha (TNF-α), interferon gamma (IFNγ) etc,

An inventive extract may be prepared in accordance with the following preferred embodiment.

For the present invention, above described extract can be prepared by follows;

The term “combined herb extract of Longanae Arillus, Ligustici Tenuissimi Rhizoma and Polygalae radix” defined herein can be prepared by the procedure comprising the steps; of slicing and washing Longanae Arillus, Ligustici Tenuissimi Rhizoma and Polygalae radix” to use as a basic extraction material at 1^{st} step; mixing together thoroughly with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50:0.1-50 weight part (w/w), more preferably, 0.1-10:0.1-10:0.1-10 weight part (w/w), more and more preferably, 1-5:1-5:1-5 weight part (w/w), most preferably, 1-3:1-3:1-3 weight part (w/w) to afford the mixed material at 2^{nd} step; adding 1-20 fold volume(v/w), preferably, 4-8 fold volume(v/w) of extracting solvent selected from the group consisting of water, C₁-C₄ lower alkyl alcohol such as methanol, ethanol, propanol, butanol, etc, acetone, ethyl acetate, chloroform, hexane, butyleneglycol, propyleneglycol or glycerin, preferably, water, methanol, ethanol, more preferably, water or 10-90% (v/v) ethanol in water, most preferably, water or 20-80% (v/v) ethanol in water to the mixed material at 3^{rd} step; extracting each solution with the extraction method by the extraction with hot water extraction, cold water extraction, reflux extraction or ultra-sonication extraction, preferably, hot water extraction at the temperature ranging from 50° C. to 120° C., preferably, about 80° C. to 100° C., for the period ranging from 1 to 24 hours, preferably, 2 to 12 hours at 4th step; repeating the above-described extraction process to collect each filtrate with filtration, drying through freeze drying, natural air drying or hot air drying process, preferably freeze drying process to obtain the combined herb extract of Longanae Arillus, Ligustici Tenuissimi Rhizoma and Polygalae radix of the present invention.

It is another object of the present invention to provide a process for preparing the combined herb extract of Longanae Arillus, Ligustici Tenuissimi Rhizoma and Polygalae radix of the present invention as described above.

It is another object of the present invention to provide an oral pharmaceutical composition comprising a combined herb extract of Longanae Arillus, Ligustici Tenuissimi Rhizoma and Polygalae radix prepared by the above-described process, as an active ingredient to treat and alleviate inflammatory diseases.

In accordance with another aspect of the present invention, there is also provided a method of treating or alleviating inflammatory diseases in a mammal comprising orally administering to said mammal an effective amount of the combined herb extract of Longanae Arillus, Ligustici Tenuissimi Rhizoma and Polygalae radix and pharmaceutically acceptable carrier thereof.

In accordance with the other aspect of the present invention, there is also provided a use of the combined herb extract of Longanae Arillus, Ligustici Tenuissimi Rhizoma and Polygalae radix for manufacture of oral preparation employed for treating or alleviating inflammatory diseases in mammals including human as an active ingredient.

It is still another object of the present invention to provide a pharmaceutical composition or health functional food comprising the herb extract of the above-mentioned herb obtained by the above described process as an active ingredient for preventing and treating inflammatory diseases.

The present inventors demonstrated that the anti-inflammatory/anti-rheumatic effects of inventive composition is potent by accomplishing in vitro experiments such as the inhibitory test on the expression of cytokines involved in inflammation (RPLPO, TSLP, GM-CSF and IL-1beta) (Experimental Example 1); Cell viability test on HT-29 and THP-1 cell (in vitro Experimental Example 2); Anti-inflammatory activity in THP-1 cell (in vitro, Experimental Example 3); inhibitory effect on autophagy activity (in vitro, Experimental Example 4.) as well as in vivo experiments such as inhibitory effect on arthritis using by arthritis-induced rat animal model (in vivo, Experimental Example 5), therefore, it is confirmed that inventive combined extract is very useful in the alleviation or treatment of inflammatory disease and arthritis disease as a form of oral pharmaceutical composition.

The pharmaceutical composition for treating purposed diseases could contain about 0.01 to 99 w/w % the above herb extract of the present invention based on the total weight of the composition.

However, the amount and each component of the above-mentioned composition can be varied with the patient's condition, development of patient's disease, the sort of disease etc.

The inventive composition may additionally comprise conventional carrier, adjuvants or diluents in accordance with a using method.

The herb composition according to the present invention can be formulated in oral dosage form such as powder, granule, tablet, capsule, suspension, emulsion, syrup, aerosol and the like; topical preparation; or injection solution. The herb composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, magnesium stearate and mineral oil. The formulations may additionally include excipients such as fillers, bulking agents, binders, wetting agents, disintegrating agents, surfactants, diluents and the like. The solid oral dosage form comprises tablet, pill, powder, granule, capsule and the like and the solid oral dosage form is prepared by adding at least one excipient such as starch, calcium carbonate, sucrose, lactose or gelatin and the like to the herb extract. Lubricant such as magnesium stearate or talc may be used. The aqueous oral dosage form comprises suspension, oral solution, emulsion, syrup and the aqueous oral dosage form may comprise several excipients such as wetting agents, sweetener flavoring agents, preservatives, as well as water, liquid paraffin. The parenteral dosage form comprises sterilized aqueous solution, non-aqueous solvent, suspension, emulsion, lyophilized preparation, suppository, and the like. Suitable examples of the carriers include propylene glycol, polyethylene glycol, vegetable oils such as olive oil, injectable ester such as ethyl oleate. Base for suppository may include witepsol, macrogol, tween 61, cacao butter, laurin, glycerogelatine etc., but are not limited to them.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01 mg/kg to 10 g/kg, preferably, 1 mg/kg to 1 g/kg by weight/day of the inventive composition of the present invention. The dose may be administered in a single or multiple doses per day.

The pharmaceutical composition of present invention can be administered to a subject animal such as mammals (rat, mouse, domestic animals or human) via various routes. All modes of administration are contemplated, for example, administration can be made orally, rectally or by intravenous injection.

In accordance with one aspect of the present invention, there provided a health functional food comprising a combined herb extract of Longanae Arillus, Ligustici Tenuissimi Rhizoma and Polygalae radix, as an active ingredient to prevent or improve inflammatory diseases.

The term “a health functional food” defined herein comprises the functional food having enhanced functionality such as physical functionality or physiological functionality by adding the extract of the present invention to conventional food to prevent or improve the purposed diseases in human or mammal and stipulated by the Law for Health Functional Foods 6727 in Republic of Korea.

The health functional food composition for preventing and improving purposed diseases could contain about 0.01 to 95 w/w %, preferably 1 to 80 w/w % of the above herb composition of present invention based on the total weight of the composition.

Moreover, the inventive extract of the present invention also can be used as a main component or additive and aiding agent in the preparation of various functional health food and health supplement food for the prevention or improvement of inflammatory diseases.

The inventive health functional food may be prepared and processed by the form of pharmaceutically acceptable dosage form such as powder, granule, tablet, capsule, pills, suspension, emulsion, syrup and the like; or the functional health food form such as tea bag, leached tea, health beverage type and the like.

It is the other object of the present invention to provide a health supplement food comprising a combined herb extract of Longanae Arillus, Ligustici Tenuissimi Rhizoma and Polygalae radix, as an active ingredient or as a main component to prevent or improve inflammatory diseases.

The above-mentioned term “as a main component” means that the above health supplement food comprises about 30 to 99 (w/w %), preferably 50 to 99 (w/w %), more preferably 70 to 99 (w/w %) of the inventive extract of present invention based on the total weight of the composition.

When the combined herb extract of the present invention is used as a component in the health functional beverage composition, the health functional beverage composition can comprise other component such as flavoring agent or natural carbohydrate without limits like that typical beverage composition. Examples of the natural carbohydrate comprise monosaccharide such as glucose, fructose etc; disaccharide such as maltose, sucrose etc; and polysaccharide, for example, sugar such as dextrin, cyclodextrin, and sugar alcohol such as xylitol, sorbitol, erythritol. Natural flavoring agent (thaumatin, stevia extract (rebaudioside A, glycyrrhizin, etc)) and synthetic flavoring agent (saccharin, aspartame, etc) may be added in the health functional beverage composition. The amount of natural carbohydrate generally ranges from about 1 to 20 g, preferably about 5 to 12 g per 100 ml of the present composition.

When the combined herb extract of the present invention is used as a food additive of the health food, the combined herb extract may be added intact or used with other food ingredient according to general process. Examples of the food comprises meat products, sausage, bread, chocolate, candy, snack, cracker, biscuit, pizza, ramen, noodle products, chewing gum, dairy products such as ice cream, soup, beverage, tea, drinks, alcohol drink, vitamin complex etc, but not intended herein to limit thereto, for preventing or improving of purposed disease.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Also, above described extract can be added to food or beverage for prevention and improvement of purposed disorder. The amount of above described extract in food or beverage as a functional health food or health supplement food may generally range from about 0.01 to 15 w/w % of total weight of food for functional health food composition. And the extract of the present invention may be added 0.02 to 5 g, preferably 0.3 to 1 g per 100 ml in health beverage composition.

### Advantageous Effects of Invention

As described in the present invention, the present inventors demonstrated that anti-inflammatory/anti-rheumatic effects of inventive combined composition is potent by accomplishing in vitro experiments such as the inhibitory test on the expression of cytokines involved in skin inflammation (RPLPO, TSLP, GM-CSF and IL-1beta) (Experimental Example 1); Cell viability test on HT-29 and THP-1 cell (in vitro Experimental Example 2); Anti-inflammatory activity in THP-1 cell (in vitro, Experimental Example 3); inhibitory effect on autophagy activity (in vitro, Experimental Example 4.) as well as in vivo experiments such as inhibitory effect on arthritis using by arthritis-induced rat animal model (in vivo, Experimental Example 5), therefore, it is confirmed that inventive combined extract is very useful in the alleviation or treatment of inflammatory disease and arthritis disease as a form of oral pharmaceutical composition.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Examples and Experimental Examples are intended to further illustrate the present invention without limiting its scope.

### Example 1. The Preparation of Inventive Combined Extract (1)

20 g of dried Longanae Arillus (Buyoung Yakup Co., Ltd.), 20 g of dried Ligustici Tenuissimi Rhizoma (Buyoung Yakup Co. Ltd.) and 20 g of dried Polygalae radix(Buyoung Yakup Co. Ltd.) were cut into small pieces, mixed with 6 fold volume (v/w) of 20% ethanol in water and the mixture was subjected to reflux extraction at 90±5° C. for 3 days. After filtration of the extract through filter paper (pore size, less than 10 μm) to remove the debris, the remaining debris was further extracted two times with 4 fold volume (v/w) of 20% ethanol in water and the extract was filtered with filter paper (pore size, less than 10 μm).

The collected extract was mixed with together and concentrated under vaccuo (16-21 Brix) to afford concentrated extract. The concentrated extract was dried with freeze drying process and pulverized (less than 50 mesh) to obtain 20.5 g (powder as dried basis, yield 33.4%) of inventive combined extract (1) (designated as “WIN-1001X” hereinafter)

### Example 2-6. The Preparation of Inventive Combined Extract (2)-(6)

Excepting adopting different combined ratio as well as different solvents disclosed in Example 1, all the procedure was identical with those in Example 1 to obtain various inventive combined extract of Longanae Arillus (LA), Ligustici Tenuissimi Rhizoma (LT) and Polygalae radix (PR) i.e., inventive combined extract (2) to inventive combined extract (6) of the present invention, which are used as a test samples in following experiment.

TABLE 1
various kinds of combined extract

| | Sample weight (g) | | | | | Extract | Final |
|---|---|---|---|---|---|---|---|
| Example | LA* | PR* | LT* | solvent* | name | weight | yield |
| Example 2 | 10 | 5 | 50 | 10% | WIN-1002X | 16.6 g | 25.6% |
| | | | | EtOH | | | |
| Example 3 | 20 | 50 | 5 | Water | WIN-1003X | 24.7 g | 32.9% |
| Example 4 | 10 | 80 | 20 | 70% | WIN-1004X | 32.3 g | 29.4% |
| | | | | BuOH | | | |
| Example 5 | 5 | 50 | 20 | 50% | WIN-1005X | 21.5 g | 28.7% |
| | | | | EtOH | | | |
| Example 6 | 30 | 10 | 2 | hexane | WIN-1006X | 12.6 g | 30.1% |

*Longanae Arillus (LA), Ligustici Tenuissimi Rhizoma (LT), Polygalae radix (PR)

### Experimental Example 1. Inhibitory Effect on Cytokine Expression (In Vitro)

In order to determine the anti-inflammatory activity of inventive extract, following inhibition test of cytokine expression using HaCaT cell was performed according to the procedure disclosed in the literature (Jeong et al., 2019, J. Invest. Dermatol., May; 139 (5): pp 1098-1109).

HaCaT cell (human epithelial keratinocyte cell, 300493, CLS) was inoculated into DMEM medium containing 10% Fetal bovine serum, 100 units/ml of penicillin, 100 μg/ml of streptomycin (D6429, Sigma-Aldrich Co. Ltd) and was incubated in the incubator (HERA cell 150i, Thermo Fisher Scientific Co. Ltd.) maintaining optimum humidity (85-95%) and 5% CO₂ atmosphere.

For performing gene expression test, the incubated cells were transferred to 12 wells and 50 ng/ml of TNF alpha (RC214-12, Biobasic Co. Ltd) was treated therewith for 1 hour to induce inflammatory response. Dexamethasone (200 nM, positive control, “DEX”, D4902, Sigma-Aldrich Co. Ltd.) and distilled water (negative control, “DIW”) were used as comparative controls.

1 hour after inducing the inflammation, 1 μg/ml of inventive extract prepared in Examples was treated with identical medium and subjected to incubation for 1 hour. After the incubation, RNA (FATRR-001, Favorgen) was extracted from the cell and cDNA was synthesized from the RNA using by cDNA synthesis kit (RRO36A, TAKARA). The polymerization reaction was performed using by the synthesized cDNA and Sybrgreen kit (RT500M, Enzynomics) and then Real-time-PCR was performed using by primers for various cytokines involved in skin inflammation (RPLPO, TSLP, GM-CSF and IL-1beta) as disclosed in Table 2.

TABLE 2
The used primers in RT-PCR method

| human* | direction | sequence | Sequence I.D |
|---|---|---|---|
| RPLP0 | forward | 5′-AGC CCA GAA CAC TGG TCT | 1 |
| | | C-3′ | |
| | reverse | 5′-ACT CAG GAT TTC AAT GGT | 2 |
| | | GCC-3′ | |
| TSLP | forward | 5′-TAT GAG TGG GAC CAA AAG | 3 |
| | | TAC CG-3′ | |
| | reverse | 5′-GGG ATT GAA GGT TAG GCT | 4 |
| | | CTG G-3′ | |
| GM- | forward | 5′-TCC TGA ACC TGA GTA GAG | 5 |
| CSF | | ACA C-3′ | |
| | reverse | 5′-TGC TGC TTG TAG TGG CTG | 6 |
| | | G-3′ | |
| IL-1β | forward | 5′-CTC CAG GGA CAG GAT ATG | 7 |
| | | GA-3′ | |
| | reverse | 5′-TCT TTC AAC ACG CAG GAC | 8 |
| | | AG-3′ | |

* abbreviation-RPLP0 (Ribosomal Protein Lateral Stalk Subunit P0); TSLP (thymic stromal lymphopoietin); GM(Granulocyte-macrophage)-CSF (colony stimulating factor); IL (interleukin)

As can be seen in Table 3 showing quantitative result of the RT-PCR, the test sample group treated with the inventive extract, sharply inhibited the expressed level of various cytokine involved in inflammation comparing with negative control group treated with distilled water (DIW) and it has been confirmed that the inhibitory activity of the test sample on the expression of various cytokine involved in inflammation is equivalent to that of positive control group treated with dexamethasone (DEX).

Accordingly, it has been confirmed that the various kind of inventive combined extract prepared in Examples 1-6 have potent inhibitory effect on inflammation.

TABLE 3
Inhibition effect on cytokine expression

| | | | | | | |
|---|---|---|---|---|---|---|
| | | TNFα | TNFα | TNFα | TNFα | |
| — | TNFα | WIN- | WIN- | WIN- | WIN- | TNFα |
| — | DIW | 1001X | 1002X | 1003X | 1005X | Dex |

TSLP

| | | | | | | |
|---|---|---|---|---|---|---|
| 1 | 132.4692 | 47.43735 | 60.85783 | 48.9323 | 55.34286 | 52.49334 |
| 0.462769 | 26.91228 | 9.645089 | 24.95619 | 19.85678 | 26.59252 | 11.2336 |

GM-CSF

| | | | | | | |
|---|---|---|---|---|---|---|
| 1 | 4.473627 | 1.982161 | 2.069408 | 2.384771 | 1.917569 | 1.935997 |
| 0.111 | 0.817826 | 0.889233 | 0.326074 | 0.871501 | 0.599711 | 0.581338 |

IL-1β

| | | | | | | |
|---|---|---|---|---|---|---|
| 1 | 4.152715 | 1.407169 | 1.437399 | 2.064964 | 1.662578 | 1.080503 |
| 0.483565 | 1.087056 | 0.394622 | 0.1926 | 0.620225 | 0.193175 | 0.413136 |

### Experimental Example 2. Cell Viability Test on HT-29 and THP-1 Cell (In Vitro)

In order to confirm the cytotoxicity of inventive extract on HT-29 cell and THP-1 cell, following cell viability using HT-29 cell and THP-1 cell was according to the previous known procedure in the art.

2-1. Procedure

HT-29 cell (human colon epithelial cell, Korean Cell Line Bank, Korean Cell Line Research Foundation, 101, Daehak-ro, Jongno-gu, Seoul, 03080, Korea) was inoculated into DMEM medium containing 10% Fetal bovine serum and 1% penicillin-streptomycin solution and THP-1 cell (human monocyte cell, Korean Cell Line Bank, Korean Cell Line Research Foundation, 101, Daehak-ro, Jongno-gu, Seoul, 03080, Korea) was inoculated into RPMI medium containing 10% Fetal bovine serum and 1% penicillin-streptomycin solution to incubate.

25, 50, 100, 250, 500 and 1000n/mL of test samples were added to HT-29 cell and THP-1 cell. 24 hours after the incubation, CCK-8 (cell counting kit-8, Dojindo Molecular Technologies, Inc.) was added thereto and the absorbance (optical density) was determined at 450 nm to determine cell viability.

2-2. Test Result

As can be seen in Table 4, it has been confirmed that the cell viability of test sample group treated with the inventive extract (25-1000 μg/mL) in HT-29 cell showed similar to that of negative control group treated with only medium and that in THP-1 cell showed 90.5% and 26.5% at the concentration of 500 and 1000 μg/mL, respectively, which showed some different with that of negative control group and less than 500 μg/mL of test sample was applied to following test.

TABLE 4
cell viability test result

| WIN1001X | | |
|---|---|---|
| μg/mL | HT-29 cell | THP-1 cell |
| 0 | 100% | 100% |
| 25 | 100.5 ± 0.6% | 102.2 ± 1.1% |
| 50 | 103.3 ± 3.2% | 103.7 ± 1.5% |
| 100 | 105.0 ± 1.4% | 104.4 ± 1.6% |
| 250 | 109.2 ± 3.1% | 101.1 ± 0.6% |
| 500 | 105.9 ± 3.3% | 90.5 ± 0.8% |
| 1000 | 90.3 ± 0.1% | 26.5 ± 5.8% |

### Experimental Example 3. Anti-Inflammatory Activity in THP-1 Cell (In Vitro)

In order to determine the anti-inflammatory activity of inventive extract, following inhibition test of the level of pro-inflammatory cytokines using THP-1 cell was performed according to the previous known procedure in the art.

3-1. Determination on the Level of IL-1Beta

3-1-1. Test Procedure

10, 20, 50, 100, and 500 μg/mL lipopolysaccharide (LPS) was added to human monocyte cell lines (THP-1 cell Korean Cell Line Bank, Korean Cell Line Research Foundation, 101, Daehak-ro, Jongno-gu, Seoul, 03080, Korea) to prepare an inflammatory model.

24 hours after LPS treatment, the level of IL-1beta, an inflammatory cytokine, in the collected cell supernatant solution was measured.

In addition, 10, 25, 50 and 100 μg/mL of test samples were treated in THP-1 cells for four hours and LPS was treated therewith to confirm the anti-inflammatory effect of the test sample.

24 hours after LPS treatment, the level of IL-1beta, a pro-inflammatory cytokine, was measured using ELISA reader (IL-1beta/IL-1F2 Duo set ELISA, R&D Systems).

3-1-2. Test Result

As can be seen in Table 5, it has been confirmed that the test sample prepared in Examples sharply reduced the level of IL-1beta which had been increased with the dose-dependently increased LPS.

Accordingly, it has been confirmed that the inventive combined extract prepared in Examples has potent inhibitory effect on inflammatory response.

TABLE 5
Inhibition effect on IL-lbeta (pro-inflammatory cytokine)

| | IL-1β level (μg/ mL) | | | |
|---|---|---|---|---|
| WIN1001X | LPS 20 | LPS 50 | LPS 100 | LPS 500 |
| μg/mL | μg/mL | μg/mL | μg/mL | μg/mL |
| 0 | 117.7 ± 14.0 | 262.8 ± 9.0 | 284.2 ± 9.0 | 319.5 ± 10.4 |
| 10 | 71.1 ± 6.5 | 214.5 ± 5.5 | 242.1 ± 4.0 | 312.2 ± 9.3 |
| 25 | 84.9 ± 7.6 | 186.5 ± 6.9 | 213.2 ± 6.2 | 285.1 ± 9.3 |
| 50 | 68.0 ± 12.4 | 158.4 ± 5.3 | 186.4 ± 3.2 | 245.2 ± 11.0 |
| 100 | 133.4 ± 8.6 | 198.9 ± 10.3 | 233.9 ± 6.0 | 311.1 ± 8.4 |

3-2. Determination on the Level of IL-10

3-2-1. Test Procedure

10, 20, 50, 100, and 500 μg/mL of lipopolysaccharide (LPS) was added to human monocyte cell lines (THP-1 cell Korean Cell Line Bank, Korean Cell Line Research Foundation, 101, Daehak-ro, Jongno-gu, Seoul, 03080, Korea) to prepare an inflammatory model.

24 hours after LPS treatment, the level of IL-10, an inflammatory cytokine, in the collected cell supernatant solution was measured.

In addition, 10, 25, 50 and 100 μg/mL of test samples were treated in THP-1 cells for four hours and LPS was treated therewith to confirm the anti-inflammatory effect of the test sample.

24 hours after LPS treatment, the level of IL-10, a pro-inflammatory cytokine, was measured using ELISA reader (IL-10, Duo set ELISA, R&D Systems).

3-2-2. Test Result

As can be seen in Table 6, it has been confirmed that the test sample prepared in Examples sharply reduced the level of IL-10 which had been increased with the dose-dependently increased LPS.

Accordingly, it has been confirmed that the inventive combined extract prepared in Examples has potent inhibitory effect on inflammatory response.

TABLE 6
Inhibition effect on IL-10 (pro-inflammatory cytokine)

| WIN1001X | IL-10 level (pg/mL) | | |
|---|---|---|---|
| μg/mL | LPS 50 μg/mL | LPS 100 μg/mL | LPS 500 μg/mL |
| 0 | 5.3 ± 1.0 | 12.0 ± 1.8 | 14.2 ± 1.1 |
| 10 | 6.2 ± 1.6 | 7.3 ± 1.7 | 15.6 ± 1.7 |
| 25 | 10.6 ± 2.1 | 6.7 ± 1.8 | 19.4 ± 1.8 |
| 50 | 5.2 ± 1.6 | 14.0 ± 1.6 | 24.5 ± 1.0 |
| 100 | 45.5 ± 2.5 | 67.3 ± 4.3 | 87.0 ± 4.8 |

### Experimental Example 4. Inhibitory Effect on Autophagy Activity (In Vitro)

In order to determine the effect of inventive extract on the expression of Inflammatory factor in immune cell, following test using THP-1 cell was performed according to the previous known procedure in the art.

4-1. Test Procedure

In order to confirm the correlation between ant-inflammatory activity and autophagy pathway, 10, 20, 50, 100, and 500 μg/mL lipopolysaccharide (LPS) was added to human monocyte cell lines (THP-1 cell Korean Cell Line Bank, Korean Cell Line Research Foundation, 101, Daehak-ro, Jongno-gu, Seoul, 03080, Korea) to prepare an inflammatory model.

After the treatment of 10, 25, 50 and 100 μg/mL of test samples, 20 and 100 μg/mL of lipopolysaccharide (LPS) was added thereto to confirm the expression of Beclin 1 and LC3B (autophagy marker) through immuno-blotting test using anti-Beclin 1 antibody (Abcam) and LC3B-antibody (Cell Signaling), respectively.

The expressed level of Beclin 1 and LC3B was quantified by scanning the resulting photo-sensitized film with ChemiDoc™MPImagingSystem (Biorad).

4-2. Test Result

As can be seen in Table 7, it has been confirmed that the test group treated with test samples and 100 μg/mL of lipopolysaccharide (LPS), showed the dose-dependently increasing effect on the expression of LC3B-1 and Beclin1.

TABLE 7
effect on the expression of Beclin 1 and LC3B

| | LPS 20 ug/mL | | LPS 100 ug/mL | |
|---|---|---|---|---|
| WIN1001X | ratio | ratio | ratio | ratio |
| μg/mL | Beclin/β-actin | LC3/β-actin | Beclin/β-actin | LC3/β-actin |
| 0 | 0.67 | 1.18 | 0.81 | 1.35 |
| 10 | 0.76 | 0.86 | 0.63 | 1.59 |
| 25 | 0.37 | 1.05 | 0.82 | 2.49 |
| 50 | 0.41 | 2.55 | 1.39 | 2.28 |
| 100 | 0.25 | 3.47 | 1.10 | 4.57 |

### Experimental Example 5. Inhibitory Effect on Arthritis (In Vivo)

To confirm the inhibitory effect of inventive extract on arthritis, the animal model test using by arthritis-induced rat animal model, was performed according to the previous known procedure in the art.

5-1. Test Procedure

In order to evaluate the efficacy of test sample on MIA(Monosodium iodoacetate)-induced osteoarthritis rat model, following test was performed at “Joint and Immune disease T2B center(chief MD. PARK, Sung-whan) located in “The Catholic University of Korea, Seoul ST. Mary's Hospital, Seoul).

5-1-1. Test Protocol

5-1-1-1. Animal Model Test Procedure

(1) Test animals: Rats (Male Wista rats, Central Lab. Animal, Seoul, Korea, 7 to 8 weeks aged, 200 to 250 g) were bred in the well-controlled breeding room in polysulfone cage (2-3 mice per cage) maintaining the temperature of 21±2° C. and relative humidity of 50±20% with the light cycle of day/dark (08:00˜20:00) at the interval of 12 hours and acclimated to the surround environment

Test method: 3 mg/kg of Monosodium iodoacetate (MIA, 12512, Sigma, Poole, UK) was dissolved in the injection saline to re reach to 60 mg/ml concentration on the day of the experiment (day 0). After dividing each groups, the experimental animals were placed in the anesthesia chamber and anesthetized with diethyl ether. 50 μL of MIA (3 mg/body) was injected into the right knee joint through the infrapatellar ligament using a 1cc syringe (26.5 gauge) in order to inducing osteoarthritis

(2) Test sample: the inventive extract prepared in Examples

(3) Positive control: Celecoxib® (Hanlim Pharmaceutical Company, Seoul, KOREA)

(4) Treatment route: oral administration 3 days after inducing osteoarthritis (once a day)

(5) The establishment of test groups: See Table 8.

5-1-1-2. Administration Route and Test Period

(1) Administration Route

After inducing osteoarthritis with MIA, test sample treatment groups (G2, G3) were prepared by homogenizing the test samples with vehicle (saline) according to the prescribed dose and orally administrating 1 mL of test sample once a day.

The negative control group (G1) was treated with only vehicle and orally administrated once a day according to the sample schedule as the test substance administration.

The positive control group (G4) was homogenized to the vehicle according to the prescribed dose and orally administered once a day.

(2) Test Period

24 rats were divided into 4 groups per 6 rats, and the test samples and positive control substance were orally administered at a specified time.

TABLE 8
The establishment of test groups

| Group | Drug administration | Dose | n |
|---|---|---|---|
| G1 | MIA + vehicle (saline) | — | 6 |
| G2 | MIA + WIN1001X (saline) | 100 mg/kg | 6 |
| G3 | MIA + WIN1001X (saline) | 150 mg/kg | 6 |
| G4 | MIA + Celecoxib (0.5% CMC) | 30 mg/kg | 6 |

negative control group(G1), test sample group(G2, G3), positive control group (G4), n = 24

5-2. Evaluation Contents

5-2-1. Determination of Pain Threshold

5-2-1-1. Determination Method of Pain Threshold

The pain threshold (nociceptive latency, threshold) was determined by using dynamic plantar aesthesiometer (Ugo Basile, 37400, Comerio, Italy), a device that gradually increases the force on the feet of Male Wistarats over a certain period of time, according to a von Freestyle evaluation method to measure pain thresholds ((Kwon J Y et al., Sci Rep. 2018 Sep. 14; 8(1):13832)

Prior to measurement, the animal was placed in an acrylic box with a wire mesh bench and acclimated for five minutes.

The pain threshold was determined by measuring the weight showing paw withdrawal behavior which animal withdraws its feet by applying a slow force of 0 to 50 g over 10 seconds using metal filaments in the center of each animal's right hind foot.

To avoid tissue damage, the cut-off threshold was set to 50 g, and the measurement time was set to the day before the inducing day of osteoarthritis by the treatment of MIA to calculate base line value in the normal control group (G1), test sample groups (G2, G3) and positive control group (G4) and the same value was obtained in the base line (day 3).

The same value was measured before the treatment of test samples (day 3) and the value was measured once a week at a certain time by administering the test samples in the articular cavity of rats.

The test result of pain threshold was calculated according to (a) paw withdrawal latencies (sec) and (b) paw withdrawal threshold (g).

5-2-1-2. Test Result of Determination of Pain Threshold

As the test result of the pain measurement, it has been confirmed that the test sample group treated with 100 and 150 mg/kg of test samples showed potent inhibitory effect on pain comparing with negative control group treated with vehicle, dose-dependently manner. (Table 9-10)

TABLE 9
The inhibitory effect on pain of each group according to time lapse
(paw withdrawal latencies, sec)

| MIA | | WIN1001X | WIN1001X | Celecoxib |
|---|---|---|---|---|
| inducement | vehicle (s) | 100 mg/kg (s) | 150 mg/kg (s) | 30 mg/kg (s) |
| Before MIA | 16.8 ± 0.8 | 16.2 ± 0.6 | 16.5 ± 0.6 | 16.0 ± 1.5 |
| treatment | | | | |
| 3 days after | 9.4 ± 1.2 | 8.0 ± 1.1 | 9.7 ± 1.1 | 9.2 ± 2.1 |
| MIA treatment | | | | |
| 6 days after | 8.6 ± 1.0 | 11.0 ± 1.3 | 11.2 ± 1.3 | 9.9 ± 2.2 |
| MIA treatment | | | | |
| 11 days after | 9.1 ± 1.4 | 12.4 ± 2.0 | 11.4 ± 1.8 | 10.2 ± 0.9 |
| MIA treatment | | | | |
| 13 days after | 9.0 ± 1.6 | 11.4 ± 1.0 | 12.1 ± 1.5 | 10.4 ± 0.9 |
| MIA treatment | | | | |
| 17 days after | 9.5 ± 0.4 | 11.1 ± 0.4 | 13.1 ± 1.4 | 10.9 ± 1.8 |
| MIA treatment | | | | |
| 21 days after | 9.0 ± 1.6 | 12.8 ± 1.6 | 13.5 ± 1.6 | 11.9 ± 1.5 |
| MIA treatment | | | | |
| 25 days after | 9.0 ± 0.5 | 13.3 ± 0.5 | 13.9 ± 2.0 | 11.6 ± 1.0 |
| MIA treatment | | | | |

TABLE 10
The inhibitory effect on pain of each group according to
forced weight (paw withdrawal threshold, g).

| | | | | Celecoxib |
|---|---|---|---|---|
| MIA | | WIN1001X | WIN1001X | 30 |
| inducement | vehicle (g) | 100 mg/kg (g) | 150 mg/kg (g) | mg/kg (g) |
| Before MIA | 33.6 ± 1.7 | 32.5 ± 1.2 | 33.0 ± 1.2 | 33.8 ± 2.9 |
| treatment | | | | |
| 3 days after | 19.0 ± 2.4 | 16.2 ± 2.1 | 19.7 ± 2.2 | 18.6 ± 4.1 |
| MIA treatment | | | | |
| 6 days after | 17.4 ± 1.8 | 22.3 ± 2.7 | 22.6 ± 2.5 | 19.9 ± 4.3 |
| MIA treatment | | | | |
| 11 days after | 18.5 ± 2.8 | 25.0 ± 4.0 | 23.1 ± 3.6 | 20.7 ± 1.7 |
| MIA treatment | | | | |
| 13 days after | 18.3 ± 3.2 | 23.0 ± 2.0 | 24.5 ± 2.9 | 21.0 ± 1.6 |
| MIA treatment | | | | |
| 17 days after | 19.2 ± 0.8 | 22.5 ± 0.9 | 26.3 ± 2.7 | 22.0 ± 3.4 |
| MIA treatment | | | | |
| 21 days after | 18.3 ± 3.1 | 25.7 ± 3.3 | 27.3 ± 3.2 | 24.0 ± 2.9 |
| MIA treatment | | | | |
| 25 days after | 18.2 ± 1.1 | 26.7 ± 0.9 | 27.9 ± 4.0 | 23.4 ± 1.9 |
| MIA treatment | | | | |

5-2-2. Test on the Determination of Weight Bearing

5-2-2-1. Determination of Weight Bearing

In order to measure the change in weight (or weight distribution) between normal hind legs (left) and arthritis-induced hind legs (right), the load of both hind feet was determined using testing apparatus (Model 600, IITC, USA) according to the method described in the literature (Kwon J Y et al., Sci Rep. 2018 Sep. 14; 8(1):13832).

The test animal shall be correctly located in the holder and fixed so that both feet can be stepped symmetrically, since the load can be varied depending on the position of the animal's foot. The particular person performs the fixation to reduce the possible error to the maximum extent.

When each animal was correctly located in the holder, the machine was operated and the weight bearing of each group was measured twice for 5 seconds per measurement.

The mean value for each test result was digitized into the weight bearing(g) of each foot.

After obtaining a baseline value before MIA treatment (Day 0), the weight bearing in the normal control group (G1), test sample groups (G2, G3) and positive control group (G4), was determined at a certain time twice a week, 3 days before the treatment of test sample (day 3).

The test results of the weight load measurement were transformed into each weight bearing ratio according to below Math 1.

Weight ratio (%)={weight of right hind limb/(weight of right hind limb+weight of left hind limb)}×100 [Math.1]

5-2-2-2. Test Result of Weight Bearing

As can be seen in Table 11, it has been confirmed that the test sample group potently improved the balance capability of both limbs comparing with the negative control group.

TABLE 11
effect on the weight bearing

| | | | | Celecoxib |
|---|---|---|---|---|
| MIA | | WIN1001X | WIN1001X | 30 |
| inducement | vehicle (%) | 100 mg/kg (%) | 150 mg/kg (%) | mg/kg (%) |
| Before MIA | 50.6 ± 1.2 | 49.6 ± 0.8 | 50.3 ± 1.3 | 49.3 ± 0.9 |
| inducement | | | | |
| 3 days | 36.9 ± 1.8 | 37.9 ± 2.7 | 35.2 ± 2.4 | 34.7 ± 4.0 |
| after MIA | | | | |
| inducement | | | | |
| 6 days | 35.5 ± 1.5 | 38.3 ± 1.7 | 38.6 ± 1.3 | 38.5 ± 1.8 |
| after MIA | | | | |
| inducement | | | | |
| 11 days | 33.8 ± 2.9 | 41.0 ± 2.5 | 39.1 ± 3.0 | 40.0 ± 3.2 |
| after MIA | | | | |
| inducement | | | | |
| 13 days | 32.3 ± 2.8 | 43.6 ± 1.4 | 44.2 ± 1.5 | 44.5 ± 0.7 |
| after MIA | | | | |
| inducement | | | | |
| 17 days | 32.2 ± 1.7 | 42.9 ± 1.2 | 42.5 ± 1.3 | 43.9 ± 1.3 |
| after MIA | | | | |
| inducement | | | | |
| 21 days | 31.6 ± 2.0 | 40.6 ± 1.2 | 41.0 ± 1.5 | 39.7 ± 3.7 |
| after MIA | | | | |
| inducement | | | | |
| 25 days | 33.3 ± 1.8 | 42.3 ± 2.3 | 42.0 ± 1.4 | 41.7 ± 1.9 |
| after MIA | | | | |
| inducement | | | | |

5-2-3. Inhibitory Test on Bone Injury (Histological Analysis, Micro-CT)

In order to determine the inhibitory effect of inventive extract on bone injury, following histological analysis was performed according to the known method disclosed in the literature (Kwon J Y et al., Sci. Rep. 2018 Sep. 14; 8(1):13832).

5-2-3-1. Inhibitory Effect on Bone Injury Caused by Osteoarthritis (Histological Analysis, Micro-CT)

In order to determine the inhibitory effect of inventive extract on bone injury caused by osteoarthritis, the MIA and test sample were administered to the right knee joint of sacrificed rats (male Wistar rats) and fixed with formalin.

The degree of bone injury around femur area of the knee joint in each group consisting of 3 rats, was determined using X-ray source (70 kV, 142 uA, AI 0.5 mm filter, rotation step 0.6°) and animal scanner (SKYSCAN1272 ex-vivo micro-CT, Bruker micro CT, Belgium) to perform micro-CT photography at 15 μm of Pixel resolution including section formation (NRecon), section rotation (Data Viewer), data Analysis (CTAN), Volume rendering generation (CTVox), and surface rendering (CTAN+CTVol).

5-2-3-2. Test Result of Histological Analysis (Micro-CT)

As can be seen in Table 12, it has been confirmed that the test sample group treated with 100 and 150 mg/kg of test samples showed potent inhibitory effect on bone injury comparing with negative control group (vehicle group) through the test result of Micro-CT photography.

TABLE 12
inhibitory effect on bone injury (Micro-CT photography)

| | | WIN1001X | WIN1001X | Celecoxib |
|---|---|---|---|---|
| | vehicle | 100 mg/kg | 150 mg/kg | 30 mg/kg |
| Bone surface | 45.7 ± 8.9 | 59.3 ± 2.9 | 56.7 ± 2.5 | 57.5 ± 0.5 |
| (%) | | | | |

5-2-4. Inhibitory Test on Bone Injury (Histopathological Analysis)

In order to determine the inhibitory effect of inventive extract on bone injury, following histopathological analysis was performed according to the known method disclosed in the literature (Kwon J Y et al., Sci Rep. 2018 Sep. 14; 8(1):13832).

5-2-3-1. Inhibitory Effect on Bone Injury Caused by Osteoarthritis (Histopathological Analysis)

In order to determine the inhibitory effect of inventive extract on bone injury caused by osteoarthritis, the MIA and test sample were administered to the right knee joint of sacrificed rats (male Wistar rats) and the right knee joint of the rat fixed with formalin was sliced to stain with Safranin O.

The histopathological analysis was performed by photographing the femur area of knee joint (×200 fold). The test result was calculated and evaluated according to the known methods including Total Mankin score (Bulstra S K et al., 1989, Clin Orthop Relat Res: 294-302.) and OARSI score (Pritzker K. P. H. et al., 2006, Osteoarthritis Cartilage 14:13-29).

5-2-3-2. Test Result of Histopathological Analysis

As can be seen in Table 13 (total Mankin score method) and Table 14 (OARSI score), it has been confirmed that the test sample group treated with 100 and 150 mg/kg of test samples showed potent inhibitory effect on bone injury comparing with negative control group (vehicle group) through the test result of histopathological analysis

TABLE 13
inhibitory effect on bone injury (total Mankin score method)

| | | WIN1001X | WIN1001X | Celecoxib |
|---|---|---|---|---|
| | vehicle | 100 mg/kg | 150 mg/kg | 30 mg/kg |
| Total Mankin | 7.5 ± 0.8 | 2.8 ± 0.6 | 4.7 ± 0.7 | 4 ± 0.6 |
| score | | | | |

TABLE 14
inhibitory effect on bone injury (OARSI score method)

| | | WIN1001X | WIN1001X | Celecoxib |
|---|---|---|---|---|
| | vehicle | 100 mg/kg | 150 mg/kg | 30 mg/kg |
| OARSI score | 4 ± 0.4 | 1.9 ± 0.4 | 2.0 ± 0.2 | 2.4 ± 0.4 |

Statistics Analysis

All data were expressed in mean and standard deviation (mean±SD), and statistical significance verification was determined to be significant (P<0.05), using two-way ANOVA, one-way ANOVA, in GraphPad PRISM Version 5.0 (USA) analysis program.

### MODE FOR THE INVENTION

Hereinafter, the formulating methods and kinds of excipients will be described, but the present invention is not limited to them. The representative preparation examples were described as follows.

Preparation of Injection

WIN-1001X extract: 100 mg

Sodium metabisulfite: 3.0 mg

Methyl paraben: 0.8 mg

Propyl paraben: 0.1 mg

Distilled water for injection: optimum amount

Injection preparation was prepared by dissolving active component, controlling pH to about 7.5 and then filling all the components in 2e ample and sterilizing by conventional injection preparation method.

Preparation of Powder

WIN-1002X extract: 500 mg

Corn Starch: 100 mg

Lactose: 100 mg

Talc: 10 mg

Powder preparation was prepared by mixing above components and filling sealed package.

Preparation of Tablet

WIN-1003X extract 200 mg

Corn Starch 100 mg

Lactose 100 mg

Magnesium stearate optimum amount

Tablet preparation was prepared by mixing above components and entabletting.

Preparation of Capsule

WIN-1004X extract: 100 mg

Lactose: 50 mg

Corn starch: 50 mg

Talc: 2 mg

Magnesium stearate optimum amount

Tablet preparation was prepared by mixing above components and filling gelatin capsule by conventional gelatin preparation method.

Preparation of Liquid

WIN-1005X extract: 1000 mg

Sugar: 20 g

Polysaccharide: 20 g

Lemon flavor: 20 g

Liquid preparation was prepared by dissolving active component, and then filling all the components in 1000 ml ample and sterilizing by conventional liquid preparation method.

Preparation of Health Food

WIN-1001X extract: 1000 mg

Vitamin mixture: optimum amount

Vitamin A acetate: 70 g

Vitamin E: 1.0 mg

Vitamin B₁₀: 13 mg

Vitamin B₂: 0.15 mg

Vitamin B6: 0.5 mg

Vitamin B1: 20.2 g

Vitamin C: 10 mg

Biotin: 10 g

Amide nicotinic acid: 1.7 mg

Folic acid: 50 g

Calcium pantothenic acid: 0.5 mg

Mineral mixture: optimum amount

Ferrous sulfate: 1.75 mg

Zinc oxide: 0.82 mg

Magnesium carbonate: 25.3 mg

Monopotassium phosphate: 15 mg

Dicalcium phosphate: 55 mg

Potassium citrate: 90 mg

Calcium carbonate: 100 mg

Magnesium chloride: 24.8 mg

The above mentioned vitamin and mineral mixture may be varied in many ways. Such variations are not to be regarded as a departure from the spirit and scope of the present invention.

Preparation of Health Beverage

WIN-1002X extract: 1000 mg

Citric acid: 1000 mg

Oligosaccharide: 100 g

Apricot concentration: 2 g

Taurine: 1 g

Distilled water: 900 ml

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the present invention provides an oral pharmaceutical composition comprising a combined herb extract of Longanae Arillus, Ligustici Tenuissimi Rhizoma and Polygalae radix, the present inventors demonstrated that the anti-inflammatory/anti-rheumatic effects of inventive combined composition is potent by accomplishing in vitro experiments such as the inhibitory test on the expression of cytokines involved in inflammation (RPLPO, TSLP, GM-CSF and IL-1beta) (Experimental Example 1); Cell viability test on HT-29 and THP-1 cell (in vitro Experimental Example 2); Anti-inflammatory activity in THP-1 cell (in vitro, Experimental Example 3); inhibitory effect on autophagy activity (in vitro, Experimental Example 4.) as well as in vivo experiments such as inhibitory effect on arthritis using by arthritis-induced rat animal model (in vivo, Experimental Example 5), therefore, it is confirmed that inventive combined extract is very useful in the alleviation or treatment of inflammatory disease and arthritis disease as a form of oral pharmaceutical composition.

1. An oral pharmaceutical composition comprising a combined herb extract of Longanae Arillus, Ligustici Tenuissimi Rhizoma and Polygalae radix, as an active ingredient for preventing and treating inflammatory diseases selected from group of pruritus caused by dermatitis, atopic dermatitis, conjunctivitis, periodontitis, rhinitis, middle ear infection, sore throat, tonsillitis, pneumonia, gastric ulcer, gastritis, Crohn's disease, colitis, hemorrhoids, gout, rheumatoid fever, lupus, fibromyalgia, tendinitis, tenosynovitis Peritendinitis, myositis hepatitis, cystitis, nephritis, Sjogren's syndrome, chronic inflammation and acute inflammation.

2. The oral pharmaceutical composition according to claim 1, wherein said “combined herb extract” is (a) combined herb extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w); or (b) the combination of each extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w).

3. The oral pharmaceutical composition according to claim 1, wherein said extract is extracted with at least one solvent selected from water methanol, ethanol, propanol, butanol, acetone, ethyl acetate, chloroform, hexane, butyleneglycol, propyleneglycol or glycerin.

4. A health functional food comprising a combined herb extract of Longanae Arillus, Ligustici Tenuissimi Rhizoma and Polygalae radix, as an active ingredient to prevent or improve inflammatory diseases selected from group of pruritus caused by dermatitis, atopic dermatitis, conjunctivitis, periodontitis, rhinitis, middle ear infection, sore throat, tonsillitis, pneumonia, gastric ulcer, gastritis, Crohn's disease, colitis, hemorrhoids, gout, rheumatoid fever, lupus, fibromyalgia, tendinitis, tenosynovitis Peritendinitis, myositis hepatitis, cystitis, nephritis, Sjogren's syndrome, chronic inflammation and acute inflammation.

5. The health functional food according to claim 4, wherein said health functional food is provided as powder, granule, tablet, capsule, pill, suspension, emulsion, syrup, tea bag, leached tea, or beverage type.

6. (canceled)

7. A method of treating or alleviating inflammatory diseases selected from group of pruritus caused by dermatitis, atopic dermatitis, conjunctivitis, periodontitis, rhinitis, middle ear infection, sore throat, tonsillitis, pneumonia, gastric ulcer, gastritis, Crohn's disease, colitis, hemorrhoids, gout, rheumatoid fever, lupus, fibromyalgia, tendinitis, tenosynovitis Peritendinitis, myositis hepatitis, cystitis, nephritis, Sjogren's syndrome, chronic inflammation and acute inflammation in a mammal comprising orally administering to said mammal an effective amount of the combined herb extract of Longanae Arillus, Ligustici Tenuissimi Rhizoma and Polygalae radix and pharmaceutically acceptable carrier thereof.

8. (canceled)


SEQUENCE LISTING (8 sequences)

SEQ ID NO: 1 - DNA, 19 bp, Homo sapiens
agcccagaacactggtctc

SEQ ID NO: 2 - DNA, 21 bp, Homo sapiens
actcaggatttcaatggtgcc

SEQ ID NO: 3 - DNA, 23 bp, Homo sapiens
tatgagtgggaccaaaagtaccg

SEQ ID NO: 4 - DNA, 22 bp, Homo sapiens
gggattgaaggttaggctctgg

SEQ ID NO: 5 - DNA, 22 bp, Homo sapiens
tcctgaacctgagtagagacac

SEQ ID NO: 6 - DNA, 19 bp, Homo sapiens
tgctgcttgtagtggctgg

SEQ ID NO: 7 - DNA, 20 bp, Homo sapiens
ctccagggacaggatatgga

SEQ ID NO: 8 - DNA, 20 bp, Homo sapiens
tctttcaacacgcaggacag